(19) United States
(12) Patent Application Publication
(10) Pub. No.: US 2007/0299489 A1
(43) Pub. Date: Dec. 27, 2007

(54) Temperature and moisture management compression shirt

(76) Inventors: Sam E. Francis JR., Johnson City, TN (US); Cynthia L. Francis, Johnson City, TN (US); Justin S. Francis, Johnson City, TN (US); Kellie L. Francis, Johnson City, TN (US)

Correspondence Address: JACOBSON HOLMAN PLLC, 400 SEVENTH STREET N.W., SUITE 600, WASHINGTON, DC, 20004, US

(21) Appl. No.: 11/812,676

(22) Filed: Jun. 20, 2007

Related U.S. Application Data
(60) Provisional application No. 60/815,277, filed on Jun. 21, 2006.

(51) Int. Cl. A61F 7/00; A41D 1/00

(52) U.S. Cl. 607/108; 2/69; 607/114

(57) ABSTRACT

A temperature and moisture management garment to be worn against an individual's skin includes a shirt, a temperature modification device, and a pocket for retaining the temperature modification device on the inside or the outside of the shirt. The temperature modification device provides a source of heat or cold without connection to an outside device for helping a wearer maintain his body temperature by applying heat or cold to an area of the wearer's body. The shirt and/or the pocket permits transmission of heat and cold from the temperature modification means to the wearer. Preferably, the shirt is made of a compression fabric to provide a snug fit and hold the temperature modification device close to the wearer's skin.

### CROSS-REFERENCE TO RELATED APPLICATIONS

The present patent application is based on, and claims priority from, U.S. provisional Application No. 60/815,277, filed Jun. 21, 2006, which is incorporated herein by reference in its entirety.

### BACKGROUND OF THE INVENTION

1. Field of the Invention

The present invention relates to garments that provide temperature management for the wearer. More specifically, the invention relates to garments made from performance fabrics, and which incorporate a device for heating and/or cooling the wearer.

2. Related Art

For years there have been deaths caused from overheating the human body either by play such as sports, working on any job in the heat, merely walking as a general exercise, sitting, relaxing to watch some sporting event such as at the speed track in the hot sun.

It has been known for ages that the core body temperature needs to be somewhat controlled to help prevent sunstroke and heatstroke to a human body and preventing a human being from being impaired. There is also a need to help maintain core body temperature in extreme conditions of cold, in order to prevent hypothermia. Hannibal addressed the problem of sunstroke and heatstroke by having runners go to the Alps to bring back snow/ice to place on the body, also have his troops simply put their feet into a mountain stream to cool their whole body. With their feet cool, the whole body would cool down to a safe body temperature thereby helping prevent heatstroke, sunstroke, etc.

Over the years, in modern times there have been various inventions to help do the same thing and that is to help cool the body down. To name a few, devices have been placed in ball caps, devices to surround the head, neck, etc. All devices have helped to some degree.

U.S. Pat. No. 5,038,779 teaches a therapeutic garment to be worn on the human torso for therapeutic treatment of back pain. The garment is provided with a pocket capable of removably receiving a packet to create a thermal change in the pocket. The distance between the pocket and the upper back section of the garment is adjustable. For these and other reasons, it is not suitable for use in the management of body temperature, particularly for athletes who must be able to wear the garment under their uniforms.

Garments disclosed in other patents for holding heating or cooling devices are bulky, and/or contain bladders, and/or require hook-up to hoses, and therefore also would not be suitable for use in the management of body temperature, particularly for athletes.

It is known that there are certain places on the body such as the feet, ankles, neck and head that if a cooling device can be used will help prevent serious problems to the body. Another strategic area is the base of the neck with the blood and nerve-endings going to and from the brain.

It is to the solution of these and other problems that the present invention is directed.

### SUMMARY OF THE INVENTION

It is accordingly a primary object of the present invention to provide a shirt that can be worn by athletes and others to manage both body temperature and moisture (perspiration).

It is another object of the present invention to provide a shirt that can help its wearer maintain core body temperature in extreme temperature conditions.

These and other objects are achieved by a shirt that places a heating or cooling device right below the neck on the top back area of the wearer.

The shirt can have long or short sleeves, or can be sleeveless. Preferably, it is made from a fabric that provides compression. The fabric also can be anti-microbial.

Other objects, features and advantages of the present invention will be apparent to those skilled in the art upon a reading of this specification including the accompanying drawings.

### BRIEF DESCRIPTION OF THE DRAWINGS

The invention is better understood by reading the following Detailed Description of the Preferred Embodiments with reference to the accompanying drawing figures, in which like reference numerals refer to like elements throughout, and in which:

FIG. 1 shows the torso of an individual wearing a temperature and moisture management shirt in accordance with the present invention, in which the pocket is located on the outside of the shirt.

FIG. 2 is an enlarged cross-sectional view of the shirt, taken along line 2-2 of FIG. 1.

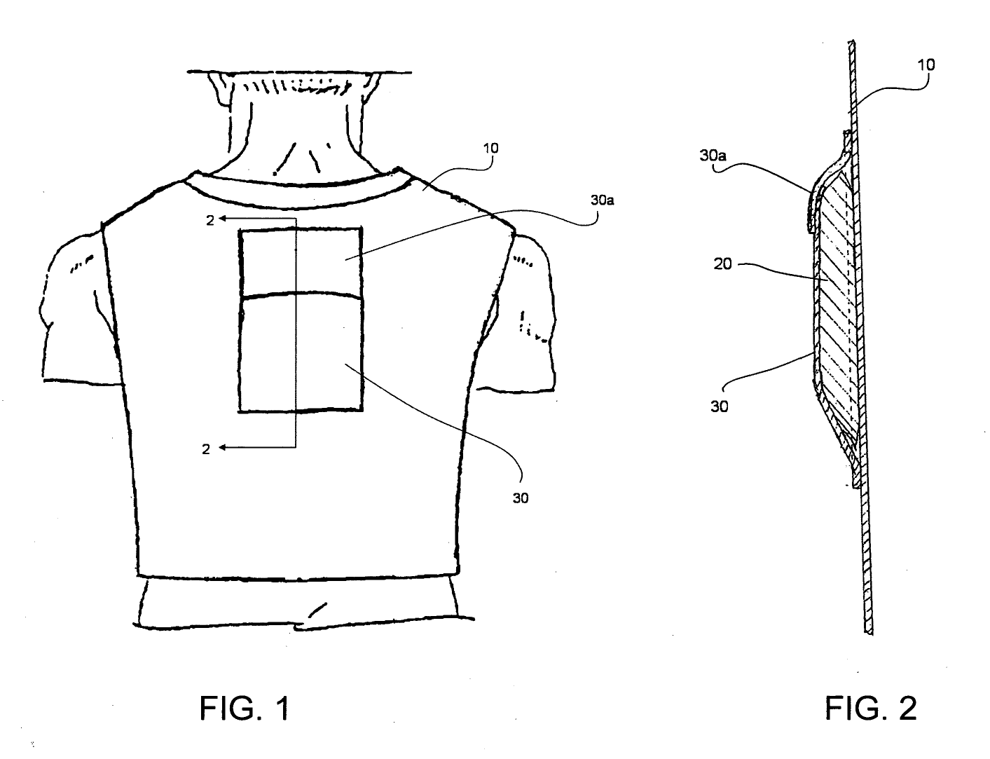

### DETAILED DESCRIPTION OF THE PREFERRED EMBODIMENTS

In describing preferred embodiments of the present invention illustrated in the drawings, specific terminology is employed for the sake of clarity. However, the invention is not intended to be limited to the specific terminology so selected, and it is to be understood that each specific element includes all technical equivalents that operate in a similar manner to accomplish a similar purpose.

The present invention is a garment, more particularly, a temperature and moisture management shirt 10 that is designed to be worn against an individual's skin, that includes a heating or cooling device 20 for respectively heating or cooling the wearer by applying heat or cold to the area below the neck at the top of the back where the blood and nerve-endings going to and from the brain are located.

The heating or cooling device 20 is retained in place by at least one pocket 30 attached securely (for example by sewing), to the inside or outside (preferably the outside) of a shirt 10, leaving one end open so that a heating or cooling device 20 can be placed in the pocket 30 and held securely next to the body. The pocket 30 thus prevents the individual from getting a burn or frost bite.

In the case where the pocket 30 is placed on the outside of the shirt 10, at least the outwardly facing surface of the pocket 30 can be made of a reflective fabric or a thin sheet of plastic material (for example, PET film) that is coated with a metallic reflecting agent, to reflects the sun's rays to help keep the packet cooler longer.

The pocket 30 also can be designed using one or more layers of material to provide additional insulation or padding or for stylistic reasons. The position of the pocket 30 is fixed; that is, its position is not adjustable. Although the pocket 30 can be located on the inside or outside of the shirt 10, it is preferably located on the outside of the shirt 10. If the pocket 30 is located on the outside of the shirt 10, the pocket 30 can have a flap 30a as a cover.

The pocket 30 can be of any shape such as rectangular, square, circular, oval, octagonal, etc. as necessary to accommodate the shape of the heating or cooling device 20. Alternatively, the pocket 30 can be sized to fit a variety of sizes of heating or cooling devices.

The pocket 30 preferably is secured to the shirt 10 permanently, but can also be detachable. Although the pocket 30 can be placed anywhere on the shirt 10, it is preferable that it be placed between the wearer's shoulder blades with the heating or cooling device 20 inserted inside. From this position, it will be helpful to send signals to the brain through the spinal cord, nerve endings and blood supplied to the brain.

The pocket 30 can be constructed as a pre-made pouch applied to the garment, thus creating two or more layers of material between the cooling/heating pack and the wearer; or a single sheet of material applied directly to the garment (i.e. by sewing), thus creating one layer of material between the wearer and the heating/cooling pack.

For most sports, the shirt 10 is worn under the uniform. However, for some sports, such as bicycling and basketball, the shirt 10 can be designed as the uniform shirt 10. It is also possible for a conventional T-shirt to be worn under the shirt 10 as is often done in the case of basketball uniforms. For use by football players, the heating or cooling device 20 must fit easily under football shoulder pads and lay flat next to the body to give maximum results for heating or cooling effects.

The pocket 30 can be designed by using any material that will be able to transmit heat or cold to the desired area, in the same way as to transmit signals to the brain which in return will transmit signals to all parts of the body through the flow of the blood or nervous system. The pocket 30 also can be of any material that absorbs perspiration into the fabric or apparel made of a material made to wick perspiration off the body.

The shirt itself can be made out of any material comfortable for contact with the skin, but some materials are more favored than others. The shirt 10 can be cotton or any type blend of material. A loose fit will not do as well, but can work. Even though the shirt 10 can have a loose fit, it is desirable that the shirt 10 have a snug fit to better hold the heating/cooling device securely in place. It is important to keep the heating or cooling device 20 firmly next to the body so the hot/cold pack can be flat as possible next to the area intended, and so that the heating or cooling device 20 is made to physically conform to the area intended, and can easily and quickly be removed from the shirt pocket 30.

The shirt 10 can be made from any material that is comfortable to the human body. Cotton and cotton blends are suitable materials. Depending upon the intended use, the shirt material can be one made to absorb moisture (such as perspiration) off an individual's body or one made to wick moisture (such as perspiration) off the body. The pocket 30 also can be made of either type of material, depending upon the intended use. The shirt 10 can be made of one of these materials while the pocket 30 can be made of the other; or the shirt 10 and pocket 30 can be made from the same material to be used for the purpose intended.

There are performance apparel product shirts that provide compression and wicks perspiration off an individual's skin as well as performance apparel that absorbs perspiration. Both fabrics are very suitable and both have benefits for the user of the shirt. As the heating or cooling device 20 must be held securely in place by the shirt 10, however the pocket 30 is designed and wherever it is located, a shirt 10 made of compression material is particularly desirable.

One very good material is Lycra® Power™, which is made by DuPont and is made of 55% nylon/45% Lycra® spandex. This is a compression product that contains a high percentage of Lycra® spandex, which can increase force and power production as well as improve accuracy of movement and is popular with athletes because an athlete is able to maintain maximum levels of performance for a longer period of time while feeling less fatigued.

Another material that could be used for the shirt 10 and pocket 30 is Adrenalink 100% polyester microfiber, a lightweight and highly breathable fabric in which the yarns spread moisture from fiber to fiber through capillary action.

The pocket material can be the same material as the shirt 10. It can be open weave, closed weave, mesh, 100% wicking polyester, or a soft textured microfiber yarn with full strength spandex and moisture wicking comfort. One side of the pocket material can be different from the other side, for any reason. For example, one side of the shirt 10 or pocket 30 can be made of material that will wick perspiration off the body with the other side of material that will absorb perspiration. If preferred, one side of the shirt 10 or pocket 30 can be made of material that will wick perspiration off the body with the other side of material that will soak up perspiration.

Whatever material is chosen for the pocket 30, the fabric must transfer heat and/or cold to make the body comfortable and help keep the temperature of the body at the desired level as much as possible. The full advantage comes in choosing the right material for the pocket 30 and shirt 10 to help give a cooling and/or heating affect to the individual.

The material should be that which is best suited for the individual wearer, whether it be for sports, watching sporting events or merely to lounge around in, as well as working in the yard, on the farm or any other job an individual does where he needs to be kept cool or warm.

The pocket material, if desirable, can be thicker than the material of the shirt itself, and can be ventilated on one or both sides or ends.

The heating or cooling device 20 is controlled by any means to help heat or cool the body, as long as the device in not of a bulky nature and is self-sufficient, so that it fits in the pocket comfortably and without being connected to some outside device. Possible heating or cooling devices 20 include a bag filled with ice or cold water (for example, from a well, creek, waterfall, or river, when ice is unavailable); a hot water bottle; Therabeads® (tiny hydroscopic beads mixed with a proprietary fluid that enhances the ability of the beads to capture moisture, and which, when heated in a microwave oven, release their retained moisture to provide pain-relieving therapeutic moist heat); super absorbent polymer pad/pack; any super saturated solvent made hot by metal disk, seed crystal, or air; pack containing buckwheat, talcum powder, rice, corn kernels and/or herbs of any kind; iron shavings activated by oxygen or any other gas; battery powered, solar powered, kinetic energy powered, steam powered, friction, static electricity, radio frequency or any other means known to man now or in the future. The pocket can be constructed so as to be liquid tight, so that ice, water, or other liquid can be placed directly into the pocket without fear of leaking.

Preference is given to gel packs that can be used for both heating and cooling by being frozen, boiled in water, or heated in a microwave; and to reusable or non-reusable instant cold packs that turn cold instantly, to reusable or non-reusable instant hot packs that turn hot instantly, and also instant gel packs. The composition of the hot/cold pack will provide the wearer from one second to twenty-four plus hours of cooling or warming time while the pack is in use next to the body.

It is highly important that the hot/cold pack be easy to insert and/or remove from the shirt pocket, depending on what the individual is wearing the cooling/heating device for. If used for sports, it is important for the wearer to be able to remove and insert the pack quickly if desired or needed during a game. A hot/cold pack, used by a sports fan watching any kind of sport, may or may not last the duration of the sporting event, and will possibly need to be changed once to several times during the event, while someone who is working or merely walking may need to change the pack but one time, if at all.

The ambient temperature can dictate how many times a hot/cold pack should be changed; however, an individual will have a chance to enjoy whatever he or she is doing on the hottest or coldest day and be as comfortable as he or she would normally be without wearing the garment in accordance with the invention.

Super absorbent polymer pellets/crystals can also be used as a heating or cooling device 20. They can be sewn into the shirt 10 or between layers of fabric or film (porous or non-porous material) and activated by soaking in water.

Modifications and variations of the above-described embodiments of the present invention are possible, as appreciated by those skilled in the art in light of the above teachings. It is therefore to be understood that, the invention may be practiced otherwise than as specifically described

What is claimed is:

1. A temperature and moisture management garment, comprising:
a shirt having an inside and an outside;
temperature modification means for providing a source of heat or cold without connection to an outside device for helping a wearer maintain his body temperature by applying heat or cold to an area of the wearer's body; and
retainer means for retaining the temperature modification means on one of the inside and the outside of the shirt;
wherein at least one of the shirt and the retainer means permits transmission of heat and cold from the temperature modification means to the wearer.

2. The garment of claim 1, wherein the retainer means is positioned to retain the temperature modification means below the wearer's neck at the top of the back.

3. The garment of claim 2, wherein the retainer means is positioned to retain the temperature modification means between the wearer's shoulder blades.

4. The garment of claim 1, wherein the retainer means is placed on the outside of the shirt and includes means for reflecting at least one of heat and light.

5. The garment of claim 1, wherein the retainer means is shaped to accommodate the shape of the temperature modification means.

6. The garment of claim 1, wherein the retainer means is shaped to accommodate temperature modification means of different sizes.

7. The garment of claim 1, wherein the retainer means is detachable from the shirt.

8. The garment of claim 1, wherein the shirt is designed as a uniform shirt for a sport.

9. The garment of claim 1, wherein the shirt is designed to be worn under sports padding.

10. The garment of claim 1, wherein the shirt is made of a material that absorbs perspiration.

11. The garment of claim 1, wherein the retainer means is made of a material that absorbs perspiration.

12. The garment of claim 1, wherein the retainer means is made of a material that wicks perspiration off the wearer's body.

13. The garment of claim 1, wherein the temperature modification means is physically conformable to the area of the wearer's body.

14. The garment of claim 1, wherein the retainer means and the temperature modification means are configured for easy insertion and removal of the temperature modification means into and from the retainer means.

15. The garment of claim 1, wherein the shirt is sized to have a snug fit.

16. The garment of claim 1, wherein the shirt and the retainer means are made of the same material.

17. The garment of claim 1, wherein the shirt and the retainer means are made of different materials.

18. The garment of claim 1, wherein the shirt is made of a material that provides compression.

19. The garment of claim 1, wherein the retainer means is on the inside of the shirt, and wherein at least the shirt permits transmission of heat or cold from the temperature modification means to the wearer.

20. The garment of claim 1, wherein the retainer means is on the inside of the shirt, and wherein the retainer means permits transmission of heat or cold from the temperature modification means to the wearer.

21. The garment of claim 1, wherein the temperature modification means includes at least one of:
a microwaveable mixture of hydroscopic beads and a fluid that enhances the ability of the beads to capture moisture;
a super absorbent polymer pad or pack;
a super saturated solvent heatable by one of metal disk, seed crystal, or air;
a pack containing at least one of buckwheat, talcum powder, rice, corn kernels, and herbs kind; and
a pack of iron shavings activated by oxygen or any other gas; battery powered, solar powered, kinetic energy powered, steam powered, friction, static electricity, radio frequency or any other means known to man now or in the future.

22. The garment of claim 1, wherein the temperature modification means is a gel pack usable for both heating and cooling.

23. The garment of claim 1, wherein the retainer means is constructed to be liquid tight.

24. The garment of claim 1, wherein the retainer means comprises a pocket attached securely to the shirt.

25. The garment of claim 24, wherein the pocket has an open end to permit insertion of the temperature modification means into the pocket.

26. The garment of claim 24, wherein the pocket is placed on the outside of the shirt, and wherein at least the outwardly facing surface of the pocket reflects at least one of heat and light.

27. The garment of claim 24, wherein the pocket comprises at least one layer of insulation or padding.

28. The garment of claim 24, wherein the pocket is constructed as a pre-made pouch applied to the shirt.

29. The garment of claim 24, wherein the pocket has inner and outer faces made of different materials.

30. The garment of claim 1, wherein the retainer means comprises layers of material sewn into the shirt, and wherein the temperature modification means comprises super absorbent polymer pellets or crystals inserted between the layers of material.

31. A temperature and moisture management garment, comprising:
a shirt having an inside and an outside; and
at least one retainer means for retaining on one of the inside and the outside of the shirt a corresponding temperature modification device that provides a source of heat or cold without connection to an outside device to help a wearer maintain his body temperature by applying heat or cold to an area of the wearer's body;
wherein at least one of the shirt and the retainer means permits transmission of heat and cold from the device to the wearer.

32. The garment of claim 31, wherein the retainer means comprises a pocket attached securely to the shirt.

33. The garment of claim 32, wherein the pocket is positioned to retain the temperature modification device below the wearer's neck at the top of the back.

34. The garment of claim 33, wherein the pocket is positioned to retain the temperature modification device between the wearer's shoulder blades.

35. The garment of claim 32, wherein the pocket is placed on the outside of the shirt and includes means for reflecting at least one of heat and light.

36. The garment of claim 32, wherein the pocket is shaped to accommodate the shape of the temperature modification device.

37. The garment of claim 32, wherein the pocket is shaped to accommodate temperature modification devices of different sizes.

38. The garment of claim 32, wherein the pocket is detachable from the shirt.

39. The garment of claim 32, wherein the shirt is designed as a uniform shirt for a sport.

40. The garment of claim 32, wherein the shirt is designed to be worn under sports padding.

41. The garment of claim 32, wherein the shirt is made of a material that absorbs perspiration.

42. The garment of claim 32, wherein the pocket is made of a material that absorbs perspiration.

43. The garment of claim 32, wherein the pocket is made of a material that wicks perspiration off the wearer's body.

44. The garment of claim 32, wherein the pocket is configured for easy insertion and removal of the temperature modification device.

45. The garment of claim 32, wherein the shirt is sized to have a snug fit.

46. The garment of claim 32, wherein the shirt and the pocket are made of the same material.

47. The garment of claim 32, wherein the shirt and the pocket are made of different materials.

48. The garment of claim 32, wherein the shirt is made of a material that provides compression.

49. The garment of claim 32, wherein the pocket is on the inside of the shirt, and wherein at least the shirt permits transmission of heat or cold from the temperature modification device to the wearer.

50. The garment of claim 32, wherein the pocket is on the inside of the shirt, and wherein the pocket permits transmission of heat or cold from the temperature modification device to the wearer.

51. The garment of claim 32, wherein the pocket is constructed to be liquid tight.

52. The garment of claim 32, wherein the pocket comprises at least one layer of insulation or padding.

53. The garment of claim 32, wherein the pocket is constructed as a pre-made pouch applied to the shirt.

54. The garment of claim 32, wherein the pocket has inner and outer faces made of different materials.
